(19) United States
(12) Patent Application Publication
(10) Pub. No.: US 2024/0346360 A1
(43) Pub. Date: Oct. 17, 2024

(54) USING QUANTUM COMPUTERS TO ACCELERATE CLASSICAL MEAN-FIELD DYNAMICS

(71) Applicant: Google LLC, Mountain View, CA (US)

(72) Inventors: Ryan Babbush, Venice, CA (US); Joonho Lee, New York, NY (US); William Huggins, Oakland, CA (US)

(21) Appl. No.: 18/510,020

(22) Filed: Nov. 15, 2023

Related U.S. Application Data
(60) Provisional application No. 63/425,602, filed on Nov. 15, 2022.
(60) Provisional application No. 63/432,308, filed on Dec. 13, 2022.

(51) Int. Cl. G06N 10/80

(52) U.S. Cl. CPC G06N 10/80

(57) ABSTRACT

Methods, systems, and apparatus for simulating a quantum system. In one aspect, a method includes performing a first quantized quantum algorithm on an initial quantum state to simulate time evolution of a fermionic system and generate a time evolved quantum state; and measuring, by the quantum computer, the time evolved quantum state to obtain one or more reduced density matrices in first quantization, the measuring including performing a classical shadows method that applies separate random Clifford channels to each qubit register of multiple qubit registers that represent respective occupied orbitals in the fermionic system.

### CROSS-REFERENCE TO RELATED APPLICATIONS

This application claims the benefit under 35 U.S.C. § 119 (e) of U.S. Patent Application No. 63/425,602, entitled “USING QUANTUM COMPUTERS TO ACCELERATE CLASSICAL MEAN-FIELD DYNAMICS,” filed Nov. 15, 2022, and U.S. Patent Application No. 63/432,308, entitled “USING QUANTUM COMPUTERS TO ACCELERATE CLASSICAL MEAN-FIELD DYNAMICS,” filed Dec. 13, 2022. The entirety of each foregoing application is incorporated herein by reference in its entirety for all purposes.

### BACKGROUND

This specification relates to quantum computing and quantum simulation. In particular, but not exclusively, the specification relates to the quantum simulation of a fermionic system (for example, an electronic system), such as the quantum simulation of the time evolution of the fermionic system.

One advantage of some quantum algorithms over mean-field classical methods is the ability to perform dynamics using the compressed representation of first quantization. In first quantization, the wavefunction is encoded using η different registers (one for each occupied orbital), each of size logN (to index the basis functions comprising each occupied orbital). The space complexity of first quantized quantum algorithms is O (η logN).

Mean-field classical methods require space complexity of O (Nη log (1/ϵ)) where e is the target precision. Thus, these quantum algorithms require exponentially less space in N. Usually, the advantage of using quantum computers to encode representations of quantum systems comes from the fact that the wavefunction might be specified by a Hilbert space vector of dimension ( [symbol] ) and could require as much space to represent explicitly on a classical computer. However, this alone cannot give exponential quantum advantage in storage in N over classical mean-field methods since mean-field methods only resolve entanglement arising from anti-symmetry and do not attempt to represent wavefunction in the full Hilbert space. Instead, the scaling advantage these quantum algorithms have over mean-field methods is related to the ability to store the distribution of each occupied orbital over N basis functions, using only logN qubits. But quantum algorithms require more than the compressed representations of first quantization in order to realize a scaling advantage over classical mean-field methods. They should also have sufficiently low gate complexity in the basis size and other parameters.

### SUMMARY

This specification describes techniques for using quantum computers to simulate fermionic systems, e.g., electronic systems. For example, the techniques may be used to simulate time evolution of a fermionic system. The techniques may provide greater understanding of the properties of the fermionic system. This may improve the ability to predict or control behavior of a system under observation and/or facilitate design and calibration of systems to be constructed.

In general, one innovative aspect of the subject matter described in this specification can be implemented in a method that includes preparing an initial quantum state, comprising preparing one or more Slater determinants of a fermionic system in first quantization; performing a first quantized quantum algorithm on the initial quantum state to simulate time evolution of the fermionic system and generate a time evolved quantum state; and measuring the time evolved quantum state.

Other implementations of these aspects include corresponding computer systems, apparatus, and computer programs recorded on one or more computer storage devices, each configured to perform the actions of the methods. A system of one or more quantum computers that include physical qubits and control electronics configured to operate the physical qubits can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations preparing the one or more Slater determinants of the fermionic system in first quantization comprises, for each Slater determinant: generating a superposition of configurations of occupied orbitals in the Slater determinant, wherein electron registers storing labels of the occupied orbitals are sorted within each configuration according to a predetermined order; and applying an anti-symmetrization procedure to the superposition of configurations.

In some implementations the predetermined order comprises ascending order.

In some implementations preparing the one or more Slater determinants of the fermionic system in first quantization comprises, for each Slater determinant: initializing multiple particle registers of qubits and an ancilla register as zero; for q=1 to N, where N represents a number of basis functions used to express each occupied orbital in the fermionic system: performing a single step of unary iteration on each particle register; adding a value in qubit q to the ancilla register; using qubit q to control unary iteration on the ancilla register; writing, using the unary iteration, a value q into the ancilla register; converting control qubits in the ancilla register to one-hot unary; for each particle register of the multiple particle registers, using the control qubits and an output from the unary iteration to control a NOT on qubit q; and converting the control qubits from one-hot unary.

In some implementations writing the value q into the ancilla register comprises using CNOT gates.

In some implementations converting control qubits in the ancilla register to one-hot unary comprises using CNOT gates.

In some implementations converting the control qubits from one-hot unary comprises uses CNOT gates.

In some implementations preparing the one or more Slater determinants of the fermionic system in first quantization further comprises, for each Slater determinant, performing a sequence of Givens rotations on the qubits to convert the qubits to first quantization.

In some implementations performing the sequence of Givens rotations comprises performing layers of Givens rotations on respective qubits, wherein performing each layer of Givens rotations converts one qubit to first quantization.

In some implementations the fermionic system comprises a system of η identical fermions that occupy N>>η orbitals and the quantum state comprise a wavefunction on η registers of n=┌log N┐ qubits included in the quantum computer.

In some implementations measuring the time evolved quantum state comprises measuring the time evolved quantum state to obtain one or more reduced density matrices (RDMs) in first quantization.

In general, another innovative aspect of the subject matter described in this specification can be implemented in a method that includes converting an arbitrary quantum state of a quantum system in second quantization to a corresponding quantum state in first quantization, comprising preparing one or more Slater determinants of the quantum system in first quantization.

Other implementations of these aspects include corresponding computer systems, apparatus, and computer programs recorded on one or more computer storage devices, each configured to perform the actions of the methods. A system of one or more quantum computers that include physical qubits and control electronics configured to operate the physical qubits can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations preparing the one or more Slater determinants of the quantum system in first quantization comprises, for each Slater determinant: generating a superposition of configurations of occupied orbitals in the Slater determinant, wherein electron registers storing labels of the occupied orbitals are sorted within each configuration according to a predetermined order; applying an anti-symmetrization procedure to the superposition of configurations.

In some implementations preparing the one or more Slater determinants in first quantization comprises, for each Slater determinant: initializing multiple particle registers of qubits and an ancilla register as zero; for q=1 to N, where N represents a number of basis functions used to express each occupied orbital in the quantum system: performing a single step of unary iteration on each particle register; adding a value in qubit q to the ancilla register; using qubit q to control unary iteration on the ancilla register; writing, using the unary iteration, a value q into the ancilla register; converting control qubits in the ancilla register to one-hot unary; for each particle register of the multiple particle registers, using the control qubits and an output from the unary iteration to control a NOT on qubit q; and converting the control qubits from one-hot unary.

In some implementations the method further comprises performing a sequence of Givens rotations on the qubits to convert the qubits to first quantization.

In some implementations performing the sequence of Givens rotations comprises performing layers of Givens rotations on respective qubits, wherein performing each layer of Givens rotations converts one qubit to first quantization.

In general, another innovative aspect of the subject matter described in this specification can be implemented in a method that includes preparing an initial quantum state; performing a first quantized quantum algorithm on the initial quantum state to simulate time evolution of a fermionic system and generate a time evolved quantum state; and measuring, by the quantum computer, the time evolved quantum state to obtain one or more reduced density matrices (RDMs) in first quantization.

The methods described may include observing the fermionic system to characterize one or more properties of the fermionic system. Preparing one or more Slater determinants of the fermionic system in first quantization and/or preparing the initial quantum state may use the characterized one or more properties.

Other implementations of these aspects include corresponding computer systems, apparatus, and computer programs recorded on one or more computer storage devices, each configured to perform the actions of the methods. A system of one or more quantum computers that include physical qubits and control electronics configured to operate the physical qubits can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations measuring the time evolved quantum state to obtain one or more one-particle reduced density matrices in first quantization comprises performing a classical shadows method that applies separate random Clifford channels to each qubit register of multiple qubit registers that represent respective occupied orbitals in the fermionic system.

In some implementations the time evolved state comprises a first quantized state and wherein measuring the time evolved quantum state to obtain the one or more RDMs in first quantization comprises: computing a classical shadow of the time evolved quantum state using an ensemble comprising a tensor product of copies of a uniform distribution over qubit Clifford circuits.

In some implementations measuring the time evolved quantum state to obtain the one or more RDMs in first quantization further comprises: determining a number of samples to include in the classical shadow; sampling, the determined number of times, from the ensemble to obtain multiple unitary operators; evaluating, for each sample, expectation values of terms in a sum over a set of permutations of multiple qubit registers that represent respective occupied orbitals in the fermionic system; grouping the samples into multiple groups of a same size; averaging within each groups; and computing a median of the averages to obtain a final estimate.

In some implementations evaluating the expectation values of the terms in the sum comprises using a Gottesman-Knill theorem.

In some implementations the multiple groups comprise

$$\\ 8 log ( \frac{1}{δ} )$$

groups, wherein δ represents a predefined target probability of accuracy.

In some implementations the same size is equal to b=4 Var(\hat{d})/ϵ², wherein ϵ represents a predefined additive error and \hat{d} represents an estimator for a k-RDM.

In some implementations the estimator for the k-RDM is given by

$${\\ \hat{d} = \frac{{k}^{{}_{} k} ( η ! )}{{η}^{{}_{} k} ( η - k ) !} {\sum }_{x ∈ {R}_{k}} Tr [ \hat{ρ} {\prod }_{l = 1}^{k} \vert {i}_{l} 〉 〈 {j}_{l} \vert}_{{x}_{l}} ] ,$$

wherein η represents a number of identical fermions in the fermionic system, Rₖ represents the sum over the set of permutations of the multiple registers, Tr represents a trace operator, \hat{ρ} represents the classical shadow, and |iₗ [symbol] [symbol] jₗ|ₓ_{ₗ} represents a tensor product of |iₗ [symbol] [symbol] jₗ| on a l-th register in an xₗ element of the sum over the set of permutations with an identity operator on other registers.

In some implementations determining the number of samples to include in the classical shadow comprises computing 64e³ log (N/δ)k(2k+2e)^{k}η^{k}ϵ⁻² where e represents Euler's number, N represents a number of fermionic orbitals in the fermionic system, 1-δ represents a predefined target probability of accuracy, k represents the size of the RDMs, η represents the number of registers, and ϵ represents a predefined additive error.

In some implementations the measuring is performed in the computational basis.

In some implementations the RDMs comprise 1-RDMS and wherein to estimate the 1-RDM elements to within a predefined additive error ϵ, measuring the time evolved quantum state comprises performing a number of measurements of the order

$$\\ \tilde{O} ( \frac{η}{{ϵ}^{{}_{} 2}} ) .$$

In some implementations the RDMs comprise k-RDMS and wherein to estimate the k-RDM elements to within a predefined additive error ϵ, measuring the time evolved quantum state comprises performing a number of measurements is of the order

$$\\ \tilde{O} ( \frac{{k}^{{}_{} k} {η}^{{}_{} k}}{{ϵ}^{{}_{} 2}} ) .$$

In some implementations the fermionic system comprises a system of η identical fermions that occupy N>>η orbitals and the quantum state comprise a wavefunction on η registers of n=┌log N┐ qubits included in the quantum computer.

In general, another innovative aspect of the subject matter described in this specification can be implemented in a method that includes measuring properties of a fermionic system wavefunction prepared using a first quantized algorithm, comprising applying separate random Clifford channels to each of qubit register of multiple qubit registers that represent occupied orbitals in the fermionic system.

Other implementations of these aspects include corresponding computer systems, apparatus, and computer programs recorded on one or more computer storage devices, each configured to perform the actions of the methods. A system of one or more quantum computers that include physical qubits and control electronics configured to operate the physical qubits can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations the time evolved state comprises a first quantized state and wherein measuring the time evolved quantum state to obtain the one or more RDMs in first quantization comprises: computing a classical shadow of the time evolved quantum state using an ensemble comprising a tensor product of copies of a uniform distribution over qubit Clifford circuits.

In some implementations measuring the time evolved quantum state to obtain the one or more RDMs in first quantization further comprises: determining a number of samples to include in the classical shadow; sampling, the determined number of times, from the ensemble to obtain multiple unitary operators; evaluating, for each sample, expectation values of terms in a sum over a set of permutations of the multiple registers; grouping the samples into multiple groups of a same size; averaging within each groups; and computing a median of the averages to obtain a final estimate.

In some implementations determining the number of samples to include in the classical shadow comprises computing 64e³ log (N/δ)k(2k+2e)^{k}η^{k}ϵ⁻² where e represents Euler's number, N represents a number of fermionic orbitals in the fermionic system, 1-δ represents a predefined target probability of accuracy, k represents the size of the RDMs, η represents the number of registers, and ϵ represents a predefined additive error.

The subject matter described in this specification can be implemented in particular embodiments so as to realize one or more of the following advantages.

Quantum algorithms for simulating fermionic systems are slower than popular classical mean-field algorithms such as Hartree-Fock and density functional theory, but offer higher accuracy. Accordingly, quantum computers have been predominantly regarded as competitors to only the most accurate and costly classical methods for treating electron correlation. However, the present disclosure describes techniques that enable exact time evolution of electronic systems with exponentially less space and polynomially fewer operations in basis set size than conventional real-time time-dependent Hartree-Fock and density functional theory. The techniques include a measurement protocol that enables all elements of a k-particle reduced density matrix to be estimated with a number of samples scaling only polylogarithmically in basis set size. The techniques also provide a more efficient quantum algorithm for first quantized mean-field state preparation.

The advantages of the proposed techniques may be particularly significant at finite temperatures. For example, a class of problems to which the described techniques may be usefully applied for speedup over mean-field methods is the electronic dynamics of either warm dense matter (WDM) or hot dense matter (HDM). The WDM regime (where thermal energy is comparable to the Fermi energy) is typified by temperatures and densities that require the accurate treatment of both quantum and thermal effects. These conditions occur in planetary interiors, experiments involving high-intensity lasers, and in inertial confinement fusion experiments as the ablator and fuel are compressed into the conditions necessary for thermonuclear ignition. Ignition occurs in the hot dense matter (HDM) regime (where thermal energy far exceeds the Fermi energy). While certain aspects of these systems are conspicuously classical, they still present spectra that can be challenging to model.

Simulations in either the WDM or HDM regime typically rely on large plane wave basis sets and the inclusion of ten to one-hundred times more partially occupied orbitals per atom than would be required at lower temperatures. Often, the attendant costs are so great that it is impractical to implement real-time time-dependent density functional theory (RT-TDDFT) with hybrid functionals. Therefore, many calculations necessarily use adiabatic semi-local approximations, even on large classical high-performance computing systems. Thus, the level of practically achievable accuracy can be quite low, and the prospect of exactly simulating the dynamics on a quantum computer is particularly compelling.

The details of one or more implementations of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system for simulating quantum systems in first quantization.

FIG. 2 is a flowchart of an example process for simulating time-evolution of a fermionic system.

FIG. 3 is a flowchart of an example process for estimating reduced density matrices of a quantum state in first quantization.

FIG. 4 is a flowchart of an example process for preparing Slater determinants of a fermionic system in first quantization.

FIG. 5 depicts an example classical/quantum computer.

### DETAILED DESCRIPTION

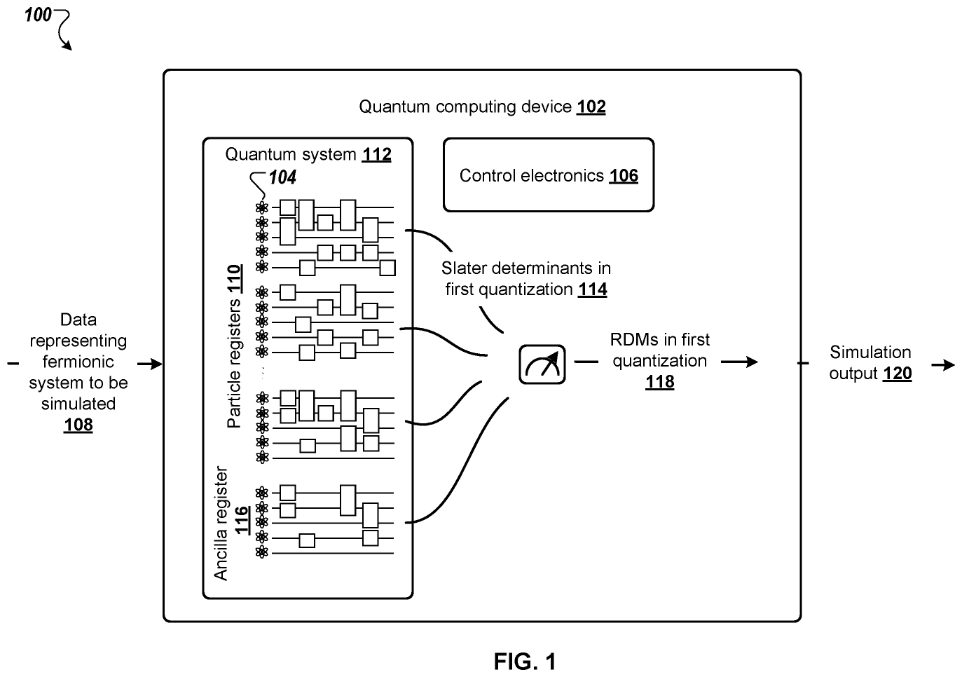

FIG. 1 is a block diagram of an example system 100 for simulating quantum systems in first quantization. The example system 100 is an example of a system implemented as classical and quantum computer programs on one or more quantum computing devices in one or more locations, in which the systems, components, and techniques described herein can be implemented.

The example computing system 100 includes a quantum computing device 102. The quantum computing device 102 includes physical components for performing classical and quantum computations. For example, the quantum computing device 102 can include a quantum data plane that, in turn, includes a quantum system 112 that includes multiple physical qubits, e.g., qubit 104, and a control and measurement system that, in turn, includes control electronics 106 that are configured to perform operations and measurements on the physical qubits.

Although not shown in FIG. 1, the quantum computing device 102 can further include a control processor plane that is configured to determine sequences of operations and measurements that a quantum algorithm being performed by the quantum computing system requires. The quantum computing device can also include a classical computer implemented as one or more computer programs, i.e., one or more modules of computer program instructions encoded on a tangible non-transitory storage medium for execution by, or to control the operation of, a data processing apparatus. The computer storage medium can be a machine-readable storage device, a machine-readable storage substrate, a random or serial access memory device, or a combination of one or more of them. The classical computer can be in data communication with the control processor, e.g., through a network, e.g., a local area network (LAN), wide area network (WAN), the Internet, or a combination thereof, and facilitates user interactions and access to networks or storage.

The particular type of the quantum computing device 102 can depend on the type of qubit used. In some implementations the qubits can be superconducting qubits, semiconducting qubits, photonic qubits, or atom-based qubits. For example, the qubits can include Xmon qubits, flux qubits, phase qubits, CAT qubits, or qubits with frequency interactions.

The example system 100 is configured to perform operations to simulate quantum systems in first quantization. For example, the quantum computing device 102 can be configured to receive data representing a fermionic system to be simulated 108. A fermionic system to be simulated can be a system of η identical fermions that occupy N>>η orbitals. In first-quantization, the state of such a system can be physically represented by the quantum computing device 102 as a wavefunction on η registers (referred to herein as particle registers 110) of n=┌log N┐ qubits (to index the basis functions comprising each occupied orbital). The wavefunction is anti-symmetric under the exchange of any two registers in order for it to represent a valid physical state. Most physically interesting observables of such fermionic systems can be captured by reduced density matrices (RDMs) of the wavefunction. Elements ^{k}Dᵢ_{₁}_{. . . i}_{ₖ}^{j}^{₁}^{. . . j}^{ₖ} of the k-body reduced density matrix (k-RDM) ^{k}D of a first-quantized state |ψ [symbol] defined on η identical fermion particles are given by Eq. (1) below.

$${}^{k} {D}_{{i}_{1} {…i}_{k}}^{{j}_{1 {… j}_{k}}} = \frac{η !}{( η - k ) !} Tr [ \vert ψ ψ \vert {\prod }_{l = 1}^{k} {\vert {i}_{l} {j}_{l} \vert}_{l} ] \qquad ( 1 )$$

In Eq. (1), i₁ . . . iₖ and j₁ . . . jₖ are indices that index the N^{2k} elements of the k-RDM, |i [symbol] [symbol] j|ₗ represents the tensor product of |i [symbol] [symbol] j| on the l-th register with an identity operator on the other η−1 registers. An equivalent definition of the elements of the k-RDM of the first-quantized state |ψ [symbol] defined on n identical fermion particles (due to the anti-symmetry of the wavefunction) is given by Eq. (2) below.

$${}^{k} {D}_{{i}_{1} {…i}_{k}}^{{j}_{1 {…j}_{k}}} = {\sum }_{x ∈ {S}_{k}^{η}}^{} Tr [ \vert ψ ψ \vert {\prod }_{l = 1}^{k} {\vert {i}_{l} {j}_{l} \vert}_{{x}_{l}} ] \qquad ( 2 )$$

In Eq. (2), Sₖ^{η} represents the set composed of all possible sequences of length k generated by drawing without replacement from [η]:={1, . . . , η}.

To perform the simulation, the quantum computing device 102 is configured to prepare the quantum system 112 in an initial quantum state and evolve the initial quantum state using a first quantized algorithm (which can be implemented through application of a quantum circuit to the initial quantum state) to obtain a time evolved quantum state in first quantization. The time evolved quantum state in first quantization can be represented by Slater determinants in first quantization 114.

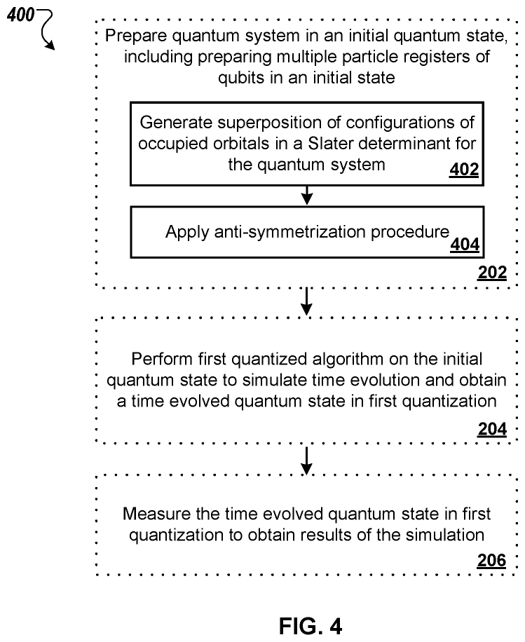

To obtain the Slater determinants in first quantization 114, the quantum computing device 102 is configured to generate a superposition of all configurations of occupied orbitals in the Slater determinant whilst ensuring that electron registers holding the label of the occupied orbitals are sorted within each configuration so that they are in ascending order. The quantum computing device 102 prepares the superposition of ordered configurations by generating the Slater determinant in second quantization in an ancilla qubit register 116 using Givens rotations, while mapping the second quantized representation to a first quantized representation one second quantized qubit (orbital) at a time. In this manner, the quantum computing device 102 only stores η non-zero qubits (orbitals) at a time in the second quantized representation because the Givens rotation algorithm gradually produces qubits that do not require further rotations. Whenever a new qubit in the second quantized representation that does not require further rotations is produced, the quantum computing device 102 converts it to the first quantized representation, which zeros that qubit. Therefore, the procedure only requires O(η) ancilla qubits and a total of O(Nη logN) gates (because for each of O(N) steps, all O(η logN) qubits of the first quantized representation are accessed). An example process for preparing Slater determinants of a fermionic system in first quantization is described in more detail below with reference to FIG. 4.

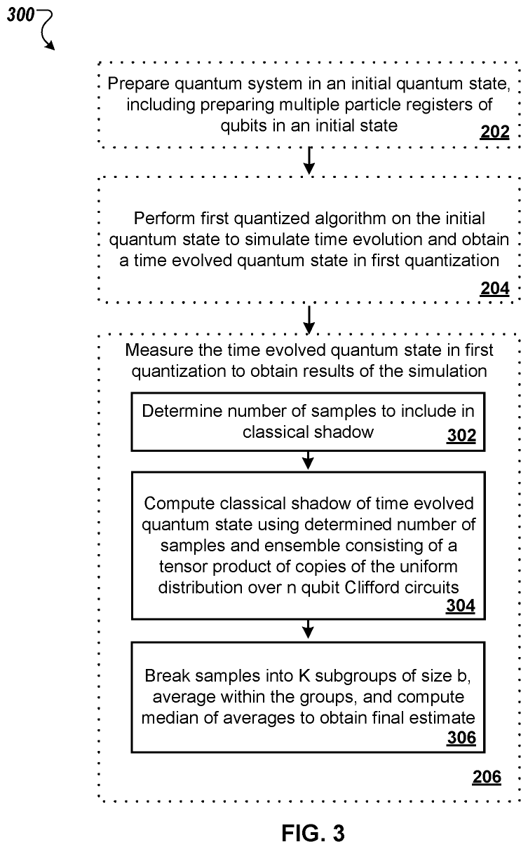

The quantum computing device 102 is then configured to implement a measurement protocol to measure the time evolved quantum state in first quantization, e.g., Slater determinants in first quantization 114, and obtain estimates of RDMs of the time evolved quantized state in first quantization 118 which can be used to generate a simulation output 120. The measurement protocol performs a classical shadows method that applies separate random Clifford channels to each log N-sized qubit register of the η registers that represent occupied orbitals in the fermionic system, and is described in more detail below with reference to FIG. 3.

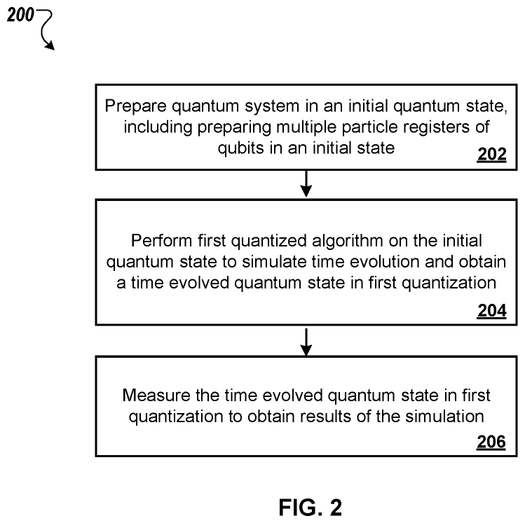

FIG. 2 is a flowchart of an example process 200 for simulating time-evolution of a fermionic system. For convenience, the process 200 will be described as being performed by components of a quantum computing system. For example, classical control electronics in communication with an array of physical qubits, e.g., the control electronics 104 of FIG. 1, appropriately programmed, can perform example process 200.

The system prepares an initial state of a quantum system (step 202). For example, for a fermionic system of η identical fermions that occupy N>>η orbitals, the system can prepare η registers of n=┌log N┐ qubits in an initial state. The system then performs a first quantized algorithm on the initial quantum state to simulate time evolution of the fermionic system and generate a time evolved quantum state (step 204). For example, the system can perform a Trotter-based first quantized algorithm or an algorithm with sublinear scaling in N, e.g., first quantized algorithms in the plane wave basis. Generally, performing the first quantized algorithm on the initial quantum state can include applying a quantum circuit to the initial state, where the quantum circuit includes quantum gates that implement a time evolution operator. Application of the quantum circuit evolves the states of the qubits included in the η registers from initial states to evolved states. The time-evolved quantum state is a first quantized quantum state (because the algorithm performed at step 204 is a first quantized algorithm).

The system then measures the time evolved quantum state, e.g., measures each qubit in the η registers, to obtain results of the simulation (step 206). For example, the system can measure the time evolved quantum state according to the operators given in Eq. (1) or (2) to obtain a classical description of the time evolved quantum state that estimates all N^{2k} elements of the k-RDM.

FIGS. 3 and 4 describe example processes that can be incorporated in one or more of steps 202-206 such that the estimates obtained at step 206 are accurate up to an additive error ϵ with probability at least 1-δ (where ϵ, δ are predetermined error thresholds) and so that example process 200 is efficient not only in terms of the number of measurements, but also in terms of the (gate) complexity of implementing each measurement and in terms of the classical complexity of the required post-processing.

### Quantum Measurement: Efficient Reduced Density Matrix Estimation Using Classical Shadows in First Quantization

FIG. 3 is a flowchart of an example process 300 for estimating reduced density matrices of a quantum state in first quantization. For example, example process 300 can be performed during step 206 of example process 200 described above with reference to FIG. 2. For convenience, the process 300 will be described as being performed by components of a quantum computing system. For example, classical control electronics in communication with an array of physical qubits, e.g., the control electronics 104 of FIG. 1, appropriately programmed, can perform example process 300.

To estimate the reduced density matrices of the quantum state in first quantization, the system performs a classical shadows method that applies separate random Clifford channels to each log N-sized qubit register of the η registers that represent occupied orbitals in the fermionic system. This measurement technique can be generally applied to efficiently characterize RDMs of wavefunctions prepared by first-quantized algorithms or simulations using a quantum computer, e.g., ground states or states obtained by time evolution from an initial configuration.

The classical shadows method is a process that can be used to estimate properties of an unknown quantum state without resorting to full state tomography (which is prohibitively expensive in terms of the number of measurements required). An ensemble of random unitaries [symbol] on n qubits is chosen, and a measurement channel given by Eq. (3) below is defined.

$$ℳ ( σ ) := {𝔼}_{U ∼ 𝒰} {\sum }_{b ∈ {\{ 0 , 1 \}}^{n}} {U}^{†} \vert b b \vert U b \vert U {σ U}^{†} \vert b \qquad ( 3 )$$

In Eq. (3), U represents a unitary operator sampled from the ensemble [symbol] , b represents a bit string of length n, and |b [symbol] represents a quantum state corresponding to bit string b.

For specific choices of [symbol] , the measurement channel [symbol] is analytically invertible. Operationally, the classical shadow of the quantum state σ is obtained by repeatedly sampling a unitary operator U from the ensemble [symbol] , applying the sampled unitary operator U to a copy of the quantum state σ, and measuring in the computational basis to obtain a bitstring b. After m such samples are collected, the quantum sate \hat{σ} given by Eq. (4) below is a classical shadow of σ.

$$\hat{σ} : = \frac{1}{m} {\sum }_{i = 1}^{m} {ℳ}^{- 1} ( {U}_{i}^{†} \vert {b}_{i} {b}_{i} \vert {U}_{i} ) \qquad ( 4 )$$

For an arbitrary observable O, an estimator ô of the quantity Tr(Oρ) using the classical shadow of ρ is defined as ô:=Tr(O\hat{ρ}). In expectation, [symbol] \hat{σ} [symbol] =σ.

To compute the classical shadow, the system uses an ensemble [symbol] that consists of a tensor product of η copies of the uniform distribution over n qubit Clifford circuits (that is, the system performs the above described measurements by independently sampling η n-qubit Clifford unitaries, applying a respect sampled n-qubit Clifford unitary to each particle register, and measuring each particle register in the computational basis). Further, to compute the classical shadow, the system obtains a number of samples m, where a maximum value of m is given by Eq. (5) below.

$$m = 64 {e}^{3} log ( N / δ ) k {( 2 k + 2 e )}^{k} {η}^{k} {ϵ}^{- 2} \qquad ( 5 )$$

In Eq. (5), e represents Euler's number, N represents the number of fermionic orbitals, 1-δ represents a predefined target probability of accuracy, k represents the size of the RDMs, η represents the number of registers, and ϵ represents a predefined additive error.

Due to the anti-symmetry of the wavefunction, there is a freedom to choose between a number of different observables when estimating the elements of the k-RDM. For example, consider an arbitrary operator O and the operator POP†, where P is an operator that permutes the particle registers. The expectation values of O and POP† with respect to a first-quantized wavefunction are the same. This degree of freedom is used to minimize the variance of the measurement protocol.

Using the observable from Eq. (1) to construct a classical shadow estimator of a k-RDM element would lead to an unnecessarily large variance, essentially because the observable does not take advantage of all of the information present in the state. In contrast, the observable from Eq. (2) defines the k-RDM element in terms of a sum over many different permutations of the registers. A measurement protocol based on the observable in Eq. (2) might perform well, but the analysis could be tedious due to the many different cases that would arise.

Therefore, rather than using the observables implied by either Eq. (1) or Eq. (2) in the classical shadow measurement procedure, the system estimates the k-RDM elements using an observable that involves a sum over a simpler set of permutations. The system partitions the η qubit registers into k groups of size η/k and measures the k-RDM element using registers from each group. For ease of notation, it is assumed that η is divisible by k (in the event that η is not exactly divisible by k, the protocol can be modified to either use groups of slightly different sizes or to only perform the measurements using η′=k└η/k┘ registers.) Consider the set of sequences given below in Eq. (6), which represents a sum over the set of permutations of the η registers.

$${R}_{k} = \{ 1 , … , \frac{η}{k} \} \times \{ \frac{η}{k} + 1 , … , \frac{2 η}{k} \} \times … \times \{ \frac{( k - 1 ) η}{k} + 1 , … , η \} \qquad ( 6 )$$

Due to the anti-symmetry of the wavefunction, the k-RDM element given in Eq. (2) can be written in terms of this set of sequences as

$${}^{k} {D}_{{i}_{1} {…i}_{k}}^{{j}_{1 {… j}_{k}}} = \frac{{k}^{k} ( η ! )}{{η}^{k} ( η - k ) !} {\sum }_{x ∈ {R}_{k}} Tr [ \vert ψ ψ \vert {\prod }_{l = 1}^{k} {\vert {i}_{l} {j}_{l} \vert}_{{x}_{l}} ] \qquad ( 7 )$$

An estimator \hat{d} for the k-RDM element ^{k}Dᵢ_{₁}_{. . . i}_{ₖ}^{j}^{₁}^{. . . j}^{ₖ} can then be defined using the classical shadow \hat{p} of the first quantized state |ψ [symbol] as given by Eq. (8) below.

$$\hat{d} = \frac{{k}^{k} ( η ! )}{{η}^{k} ( η - k ) !} {\sum }_{x ∈ {R}_{k}} Tr [ \hat{ρ} {\prod }_{l = 1}^{k} {\vert {i}_{l} {j}_{l} \vert}_{{x}_{l}} ] \qquad ( 8 )$$

The single shot variance of this estimator is bounded by Var(\hat{d})≤e³η^{k}(2k+2e)^{k}. In order to guarantee that the results of the simulation (the estimates) are close to the true value of the k-RDM elements with high probability, a median-of-means estimator is constructed to obtain the target rigorous guarantees. The system uses an estimator that divides the m total classical shadow samples into K groups of size b and takes the median of the sample mean obtained by averaging the estimates within each group. The probability that this median of means estimator has an error larger than 2\sqrt{Var(\hat{d})/b} is at most e^{\K/8}. Bounding the error by ϵ with a success probability of at least 1-δ implies that it is required that b=4 Var(\hat{d})/ϵ² and

$$K = 8 log ( \frac{1}{δ} ) .$$

The overall number of measurements given by Eq. (5) follows directly from applying a union bound over the failure probabilities for estimating all N^{2k} k-RDM elements.

Returning to the description of example process 300 and FIG. 3, the measurement protocol can be summarized as follows. The system computes a classical shadow of the time evolved quantum state (step 304). That is, the system computes a classical shadow given by Eq. (4) using an ensemble [symbol] that consists of a tensor product of η copies of the uniform distribution over n qubit Clifford circuits. The system determines a number of samples to include in the classical shadow according to Eq. (5) (step 302). For each sample, the system evaluates expectation values of the (η/k)^{k} different terms in the sum over the set of sequences given by Eq. (6), e.g., using generalizations of Gottesman-Knill theorem that account for the phase of the quantities involved. The system breaks (i.e., partitions) the samples into K groups of size b, averages within the groups, and computes the median of the averages to obtain a final estimate (step 306).

The costs associated with example process 300 can be summarized as follows. Applying a random Clifford channel on log N qubits require O(log² N) gates. Therefore, O(η log² N) gates comprise the full channel (a negligible cost relative to time-evolution). Repeating this procedure Õ(η/ϵ²) times enables estimation of all 1-RDM elements to within additive error ϵ (and, more generally, the procedure allows for estimating all higher order k-particle RDMs elements with Õ(k^{k}η^{k}/ϵ²) circuit repetitions). For example, consider the 1-RDM elements of a wavefunction |ψ(t) [symbol] in first quantization, as given by Eq. (9) below.

$${ρ}_{μ v} ( t ) = ψ ( t ) \vert ( {\sum }_{j = 1}^{η} {\vert μ v \vert}_{j} ) \vert \vert ψ ( t ) \qquad ( 9 )$$

In Eq. (9), the subscript j indicates which of the η registers the orbital-ν to orbital-μ transition operator acts upon. Due to the anti-symmetry of the occupied orbital registers in first quantization, the 1-RDM could also be obtained by measuring the expectation value of an operator such as η|p [symbol] [symbol] q|₁, which acts on just one of the η registers. Because η|p [symbol] [symbol] q|₁ has the Hilbert-Schmidt norm of O(η), the standard classical shadows procedure applied to this logN sized register would require e O(η²/ϵ²) repetitions. But the procedure can be parallelized by also collecting classical shadows on the other η−1 registers simultaneously. Due to anti-symmetry, these registers are anti-correlated. As a result, collecting shadows on all η registers simultaneously reduce the overall cost by at least a factor of η. To obtain W elements of the 1-RDM an offline classical inversion of the Clifford channel needs to be performed, which scales as Õ(Wη²/ϵ²). Any quantum or classical algorithm for estimating W quantities must have gate complexity of at least W. However, this only needs to be performed once and does not scale in t. As a comparison, the cost of computing 1-RDM classically without exploiting sparsity is Õ(Wη).

When simulating systems that are well described by mean-field theory, all observables can be efficiently obtained from the time-dependent 1-RDM. However, for observables such as energy that have a norm growing in system size or basis size, targeting fixed additive error in the 1-RDM elements will not be sufficient for fixed additive error in the observable. In such situations, it could be preferable to estimate the observable of interest directly using a combination of block encodings and amplitude amplification. Assuming the cost of block encoding the observable is negligible to the cost of time-evolution (true for many observables, including energy), this results in needing O(λ/ϵ) circuit repetitions, where λ is the 1-norm associated with the block encoding of the observable. For example, whereas there are many correlation functions with λ=O(1), for the energy

$$λ = O ( {N}^{\frac{1}{3}} {η}^{\frac{5}{3}} + {N}^{\frac{2}{3}} {η}^{\frac{1}{3}} )$$

multiplying that to the cost of quantum time-evolution further reduces the quantum speedup.

The final measurement cost to consider is that of resolving observables in time. In some cases, e.g., when computing scattering cross sections or reaction rates, it can suffice when the system measures the state of the simulation at a single point in time t. However, in other situations, it might be preferable to simulate time-evolution up to a maximum duration of t, but sample quantities at L different points in time. Most quantum simulation methods that accomplish this goal scale as O(L) (or O(Lt) in the case where the points are evenly spaced in time). However, this cost can be reduced to O(\sqrt{L}t), but with an additional additive space complexity of Õ(L).

### Quantum State Preparation: Preparing Slater Determinants within First Quantization

FIG. 4 is a flowchart of an example process 400 for preparing Slater determinants of a fermionic system in first quantization. For example, example process 400 can be performed during step 202 and/or 204 of example process 200 described above with reference to FIG. 2. For convenience, the process 400 will be described as being performed by components of a quantum computing system. For example, classical control electronics in communication with an array of physical qubits, e.g., the control electronics 104 of FIG. 1, appropriately programmed, can perform example process 400.

To prepare the Slater determinants of the fermionic system in first quantization, the system generates a superposition of all of the configurations of occupied orbitals in a Slater determinant for the fermionic system (step 402). In order to prepare such a superposition, step 402 may comprise carrying out one or more observations of the fermionic system in order to characterize its properties. Such observations may include any observable property, amongst which are the properties of the wave functions, Hamiltonians or symmetries of the system or a sample thereof. The superposition is generated to ensure that electron registers holding the label of the occupied orbitals are sorted within each configuration according to a predetermined order, e.g., so that they are in an ascending order. Then, an anti-symmetrization procedure is applied (step 404). The structure of the first step is important since without this structure (or guarantees of something similar), the second step (anti-symmetrization) could not be reversible. The method requires only O(ηN) quantum gates, where η represents the number of occupied orbitals and N represents the number of basis functions used to express each occupied orbital—which is less than conventional methods that typically require O(η²N) gates, e.g., methods based on adapting a first quantized version of Givens rotations.

This state preparation technique can be generally used to convert arbitrary states that have been prepared in second quantization into states in first quantization. To avoid needing to store all N qubits for the second quantized state as it is produced, its qubits are converted to the first quantized representation. For example, the technique could be used so that an efficient measurement technique in first quantization can be applied to a state in second quantization (by obtaining an arbitrary quantum state of a quantum system in second quantization, converting the quantum state in second quantization to a corresponding quantum state in first quantization, and measuring the quantum state in first quantization using the measurement techniques described with reference to FIG. 3).

In more detail, consider how a state in the second quantized representation can be converted to the first quantized representation. A computational basis state in second quantization consists of a string of N bits with η ones and N−η zeros. The conversion procedure is to run through these qubits in sequence and store the locations in η registers of size ┌log N┐. Let the qubit number considered from the second quantized representation be referred to as q and let the number of electrons (ones) found so far be ζ. The value of ζ will be stored in an ancilla register of size n_{η}=┌log(η+1)┐. The system initializes all η registers for the first quantized representation and the ζ register as zero. Then, for q=1 to N the system performs the following operations:
1. The system adds the value in qubit q to the ζ register, with Toffoli cost n_{η}−1. If the qubit is in the 1-state, then the system increments the value ζ.
2. The system uses qubit q to control unary iteration on the register ζ, which has cost η−1.
3. The system uses the unary iteration to write the value q into register ζ using CNOT gates. Because q is iterated classically, only CNOTs are needed, with no further Toffolis beyond that needed for the unary iteration. Because the unary iteration is controlled by qubit q, in the case where qubit q is in a 0-state, the unary iteration does not proceed and the value of q is not written out.
4. The system performs unary iteration on ζ again that is not controlled; the cost is η−2.
5. The system uses the unary iteration on ζ to check if the value in register number ζ is q; if it is then the system performs a NOT on qubit q. This multiply-controlled Toffoli is controlled by ┌log N┐+1 qubits (including the qubit from the unary iteration), so it has a cost of ┌log N┐. But this is done for each of the n registers, for a total cost η┌log N┐.
   The last operation ensures that qubit q is set to the zero state. That is because, if it is initially in the zero state, then value q is not written in register ζ, and the value is not flipped. If it is initially in the one state, then q is written in register ζ, and the multiply-controlled Toffoli flips this qubit to the zero state.

This procedure gives an ordered list of the electron positions, but an anti-symmetrized state is needed. Therefore, an anti-symmetrization procedure is applied with cost O(η logη log N). The total Toffoli cost is N(2η+n_{η}−3+η┌logN┐)+O(η logη log N). The dominant cost here is ηN log N from erasing the qubits in the second quantized representation, with the factor of log N coming from the need to check all qubits of each register to check if it is q. However, in unary iteration it is possible to check if a register is equal to a consecutive sequence of values without this logarithmic overhead, and consecutive values of q are considered here.

Therefore, to eliminate that overhead, the system performs simultaneous unary iteration on all of the η registers. That is, for each register for the first quantized representation, the system stores the qubits needed for unary iteration, as well as a control register to ensure that the system does not iterate on registers that do not have value written into them yet. The control qubits correspond to the value of ζ in unary. The modified procedure is therefore as follows (with the iteration of q from 1 to N):
1. The system performs a single step of unary iteration on all η registers with cost η Toffolis.
2. The system adds the value in qubit q to the ζ register, with Toffoli cost n_{η}−1.
3. The system uses qubit q to control unary iteration on the register ζ, which has cost η−1.
4. The system uses this unary iteration to write the value q into register ζ, as well as the ┌log N┐ ancilla qubits for the unary iteration and the control qubit. Again, this is performed with CNOT gates.
5. The system converts the control qubits to one-hot unary using a sequence of CNOT gates.
6. For each of the η registers, the system uses the control qubit and the unary iteration output to control a NOT on qubit q. This has a cost of a single Toffoli for each register, for a total of η.
7. The system converts the control qubits from one-hot unary with CNOT gates.
   As a result, the log N factor is eliminated and the cost of η−2 for the unary iteration on ζ is eliminated (because the control qubits are a unary representation of ζ). It could be argued that the binary representation of ζ is not needed; however, it would be more costly to add increment ζ in unary (about η cost instead of log η). The total Toffoli cost of this procedure is then N(3η+n_{η}−2)+O(η log η log N), where the order term is the cost for anti-symmetrizing. Note that this reduces the Toffoli cost, but there is still a Clifford cost of Nη log N from the CNOTs to place the value of q in the first quantized registers.

Then, to efficiently prepare the Slater determinant, the system performs a sequence of Givens rotations on the qubits for the second quantized representation. The Givens rotations are performed in a sequence where Givens rotations can be performed in a layer on qubits 1 to η+1, then on qubits 2 to η+2, then 3 to η+3, and so on. Generally, layer q of Givens rotations can be performed on qubits q to η+q. After the first layer there are only η+1 qubits being used, and the first qubit is not accessed again in the preparation. Therefore, the system can convert this qubit to the first quantized representation and erase it. Then there are only η qubits actively being used in the second quantized representation, and the next layer will be performed on qubits 2 to η+2, bringing on one more qubit.

In this way, each time the system performs a layer of Givens rotations to prepare the state, it can convert one qubit to the first quantized representation, and only η+1 qubits of the second quantized representation need be used at once, which is trivial compared to the number of qubits used for the first quantized representation.

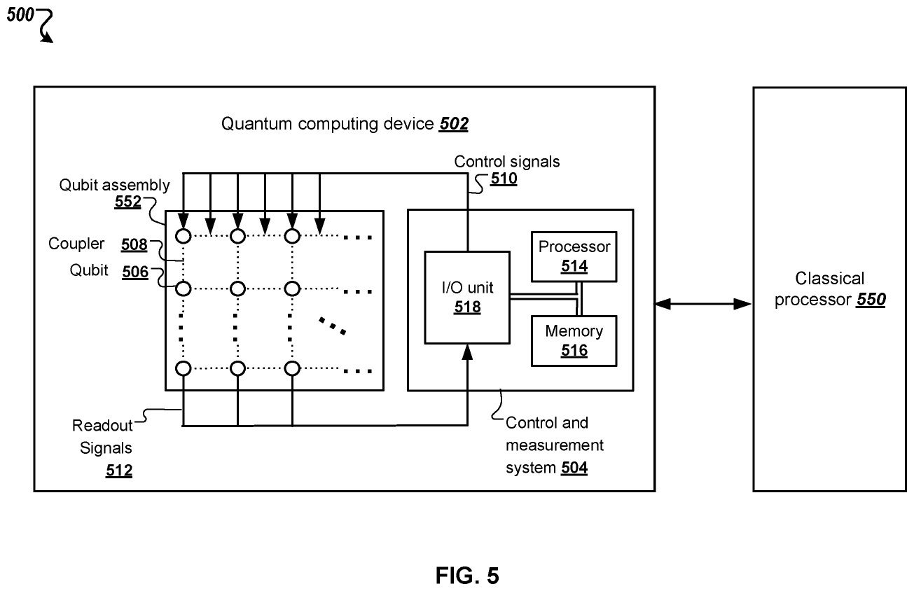

FIG. 5 depicts an example classical/quantum computer 500 for performing some or all of the classical and quantum operations described in this specification. The example classical/quantum computer 500 includes an example quantum computing device 502. The quantum computing device 502 is intended to represent various forms of quantum computing devices. The components shown here, their connections and relationships, and their functions, are exemplary only, and do not limit implementations of the inventions described and/or claimed in this document.

The example quantum computing device 502 includes a qubit assembly 552 and a control and measurement system 504. The qubit assembly includes multiple qubits, e.g., qubit 506, that are used to perform algorithmic operations or quantum computations. While the qubits shown in FIG. 5 are arranged in a rectangular array, this is a schematic depiction and is not intended to be limiting. The qubit assembly 552 also includes adjustable coupling elements, e.g., coupler 508, that allow for interactions between coupled qubits. In the schematic depiction of FIG. 5, each qubit is adjustably coupled to each of its four adjacent qubits by means of respective coupling elements. However, this is an example arrangement of qubits and couplers, and other arrangements are possible, including arrangements that are non-rectangular, arrangements that allow for coupling between non-adjacent qubits, and arrangements that include adjustable coupling between more than two qubits.

Each qubit can be a physical two-level quantum system or device having levels representing logical values of 0 and 1. The specific physical realization of the multiple qubits and how they interact with one another is dependent on a variety of factors including the type of the quantum computing device 502 included in the example computer 500 or the type of quantum computations that the quantum computing device is performing. For example, in an atomic quantum computer the qubits may be realized via atomic, molecular or solid-state quantum systems, e.g., hyperfine atomic states. As another example, in a superconducting quantum computer the qubits may be realized via superconducting qubits or semi-conducting qubits, e.g., superconducting transmon states. As another example, in a NMR quantum computer the qubits may be realized via nuclear spin states.

In some implementations a quantum computation can proceed by loading qubits, e.g., from a quantum memory, and applying a sequence of unitary operators to the qubits. In some implementations the sequence of unitary operators can represent forward or backward time evolution. Applying a unitary operator to the qubits can include applying a corresponding sequence of quantum logic gates to the qubits, e.g., to implement a quantum algorithm such as a quantum principle component algorithm. Example quantum logic gates include single-qubit gates, e.g., Pauli-X, Pauli-Y, Pauli-Z (also referred to as X, Y, Z), Hadamard gates, S gates, rotations, two-qubit gates, e.g., controlled-X, controlled-Y, controlled-Z (also referred to as CX, CY, CZ), controlled NOT gates (also referred to as CNOT) controlled swap gates (also referred to as CSWAP), and gates involving three or more qubits, e.g., Toffoli gates. The quantum logic gates can be implemented by applying control signals 510 generated by the control and measurement system 504 to the qubits and to the couplers.

For example, in some implementations the qubits in the qubit assembly 552 can be frequency tunable. In these examples, each qubit can have associated operating frequencies that can be adjusted through application of voltage pulses via one or more drive-lines coupled to the qubit. Example operating frequencies include qubit idling frequencies, qubit interaction frequencies, and qubit readout frequencies. Different frequencies correspond to different operations that the qubit can perform. For example, setting the operating frequency to a corresponding idling frequency may put the qubit into a state where it does not strongly interact with other qubits, and where it may be used to perform single-qubit gates. As another example, in cases where qubits interact via couplers with fixed coupling, qubits can be configured to interact with one another by setting their respective operating frequencies at some gate-dependent frequency detuning from their common interaction frequency. In other cases, e.g., when the qubits interact via tunable couplers, qubits can be configured to interact with one another by setting the parameters of their respective couplers to enable interactions between the qubits and then by setting the qubit's respective operating frequencies at some gate-dependent frequency detuning from their common interaction frequency. Such interactions may be performed in order to perform multi-qubit gates.

The type of control signals 510 used depends on the physical realizations of the qubits. For example, the control signals may include RF or microwave pulses in an NMR or superconducting quantum computer system, or optical pulses in an atomic quantum computer system.

A quantum computation can be completed by measuring the states of the qubits, e.g., using a quantum observable such as X or Z, using respective control signals 510. The measurements cause readout signals 512 representing measurement results to be communicated back to the control and measurement system 504. The readout signals 512 may include RF, microwave, or optical signals depending on the physical scheme for the quantum computing device and/or the qubits. For convenience, the control signals 510 and readout signals 512 shown in FIG. 5 are depicted as addressing only selected elements of the qubit assembly (i.e., the top and bottom rows), but during operation the control signals 510 and readout signals 512 can address each element in the qubit assembly 552.

The control and measurement system 504 is an example of a classical computer system that can be used to perform various operations on the qubit assembly 552, as described above, as well as other classical subroutines or computations. The control and measurement system 504 includes one or more classical processors, e.g., classical processor 514, one or more memories, e.g., memory 516, and one or more I/O units, e.g., I/O unit 518, connected by one or more data buses. The control and measurement system 504 can be programmed to send sequences of control signals 510 to the qubit assembly, e.g., to carry out a selected series of quantum gate operations, and to receive sequences of readout signals 512 from the qubit assembly, e.g., as part of performing measurement operations.

The processor 514 is configured to process instructions for execution within the control and measurement system 504. In some implementations, the processor 514 is a single-threaded processor. In other implementations, the processor 514 is a multi-threaded processor. The processor 514 is capable of processing instructions stored in the memory 516.

The memory 516 stores information within the control and measurement system 504. In some implementations, the memory 516 includes a computer-readable medium, a volatile memory unit, and/or a non-volatile memory unit. In some cases, the memory 516 can include storage devices capable of providing mass storage for the control and measurement system 504, e.g., a hard disk device, an optical disk device, a storage device that is shared over a network by multiple computing devices (e.g., a cloud storage device), and/or some other large capacity storage device.

The input/output device 518 provides input/output operations for the control and measurement system 504. The input/output device 518 can include D/A converters, A/D converters, and RF/microwave/optical signal generators, transmitters, and receivers, whereby to send control signals 510 to and receive readout signals 512 from the qubit assembly, as appropriate for the physical scheme for the quantum computer. In some implementations, the input/output device 518 can also include one or more network interface devices, e.g., an Ethernet card, a serial communication device, e.g., an RS-232 port, and/or a wireless interface device, e.g., an 802.11 card. In some implementations, the input/output device 518 can include driver devices configured to receive input data and send output data to other external devices, e.g., keyboard, printer and display devices.

Although an example control and measurement system 504 has been depicted in FIG. 5, implementations of the subject matter and the functional operations described in this specification can be implemented in other types of digital electronic circuitry, or in computer software, firmware, or hardware, including the structures disclosed in this specification and their structural equivalents, or in combinations of one or more of them.

The example system 500 also includes an example classical processor 550. The classical processor 550 can be used to perform classical computation operations described in this specification according to some implementations.

Implementations of the subject matter and operations described in this specification can be implemented in digital electronic circuitry, analog electronic circuitry, suitable quantum circuitry or, more generally, quantum computational systems, in tangibly-embodied software or firmware, in computer hardware, including the structures disclosed in this specification and their structural equivalents, or in combinations of one or more of them. The term “quantum computational systems” may include, but is not limited to, quantum computers, quantum information processing systems, quantum cryptography systems, or quantum simulators.

Implementations of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, one or more qubits, or a combination of one or more of them. Alternatively, or in addition, the program instructions can be encoded on an artificially-generated propagated signal that is capable of encoding digital and/or quantum information, e.g., a machine-generated electrical, optical, or electromagnetic signal, that is generated to encode digital and/or quantum information for transmission to suitable receiver apparatus for execution by a data processing apparatus.

The terms quantum information and quantum data refer to information or data that is carried by, held or stored in quantum systems, where the smallest non-trivial system is a qubit, i.e., a system that defines the unit of quantum information. It is understood that the term “qubit” encompasses all quantum systems that may be suitably approximated as a two-level system in the corresponding context. Such quantum systems may include multi-level systems, e.g., with two or more levels. By way of example, such systems can include atoms, electrons, photons, ions or superconducting qubits. In many implementations the computational basis states are identified with the ground and first excited states, however it is understood that other setups where the computational states are identified with higher level excited states are possible.

The term “data processing apparatus” refers to digital and/or quantum data processing hardware and encompasses all kinds of apparatus, devices, and machines for processing digital and/or quantum data, including by way of example a programmable digital processor, a programmable quantum processor, a digital computer, a quantum computer, multiple digital and quantum processors or computers, and combinations thereof. The apparatus can also be, or further include, special purpose logic circuitry, e.g., an FPGA (field programmable gate array), an ASIC (application-specific integrated circuit), or a quantum simulator, i.e., a quantum data processing apparatus that is designed to simulate or produce information about a specific quantum system. In particular, a quantum simulator is a special purpose quantum computer that does not have the capability to perform universal quantum computation. The apparatus can optionally include, in addition to hardware, code that creates an execution environment for digital and/or quantum computer programs, e.g., code that constitutes processor firmware, a protocol stack, a database management system, an operating system, or a combination of one or more of them.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. A quantum computer program, which may also be referred to or described as a program, software, a software application, a module, a software module, a script, or code, can be written in any form of programming language, including compiled or interpreted languages, or declarative or procedural languages, and translated into a suitable quantum programming language, or can be written in a quantum programming language, e.g., QCL or Quipper.

A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a digital and/or quantum data communication network. A quantum data communication network is understood to be a network that may transmit quantum data using quantum systems, e.g. qubits. Generally, a digital data communication network cannot transmit quantum data, however a quantum data communication network may transmit both quantum data and digital data.

The processes and logic flows described in this specification can be performed by one or more programmable computers, operating with one or more processors, as appropriate, executing one or more computer programs to perform functions by operating on input data and generating output. The processes and logic flows can also be performed by, and apparatus can also be implemented as, special purpose logic circuitry, e.g., an FPGA or an ASIC, or a quantum simulator, or by a combination of special purpose logic circuitry or quantum simulators and one or more programmed digital and/or quantum computers.

For a system of one or more computers to be “configured to” perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. For example, a quantum computer may receive instructions from a digital computer that, when executed by the quantum computing apparatus, cause the apparatus to perform the operations or actions.

Computers suitable for the execution of a computer program can be based on general or special purpose processors, or any other kind of central processing unit. Generally, a central processing unit will receive instructions and data from a read-only memory, a random access memory, or quantum systems suitable for transmitting quantum data, e.g. photons, or combinations thereof.

The elements of a computer include a central processing unit for performing or executing instructions and one or more memory devices for storing instructions and digital, analog, and/or quantum data. The central processing unit and the memory can be supplemented by, or incorporated in, special purpose logic circuitry or quantum simulators. Generally, a computer will also include, or be operatively coupled to receive data from or transfer data to, or both, one or more mass storage devices for storing data, e.g., magnetic, magneto-optical disks, optical disks, or quantum systems suitable for storing quantum information. However, a computer need not have such devices.

Quantum circuit elements (also referred to as quantum computing circuit elements) include circuit elements for performing quantum processing operations. That is, the quantum circuit elements are configured to make use of quantum-mechanical phenomena, such as superposition and entanglement, to perform operations on data in a non-deterministic manner. Certain quantum circuit elements, such as qubits, can be configured to represent and operate on information in more than one state simultaneously. Examples of superconducting quantum circuit elements include circuit elements such as quantum LC oscillators, qubits (e.g., flux qubits, phase qubits, or charge qubits), and superconducting quantum interference devices (SQUIDs) (e.g., RF-SQUID or DC-SQUID), among others.

In contrast, classical circuit elements generally process data in a deterministic manner. Classical circuit elements can be configured to collectively carry out instructions of a computer program by performing basic arithmetical, logical, and/or input/output operations on data, in which the data is represented in analog or digital form. In some implementations, classical circuit elements can be used to transmit data to and/or receive data from the quantum circuit elements through electrical or electromagnetic connections. Examples of classical circuit elements include circuit elements based on CMOS circuitry, rapid single flux quantum (RSFQ) devices, reciprocal quantum logic (RQL) devices and ERSFQ devices, which are an energy-efficient version of RSFQ that does not use bias resistors.

In certain cases, some or all of the quantum and/or classical circuit elements may be implemented using, e.g., superconducting quantum and/or classical circuit elements. Fabrication of the superconducting circuit elements can entail the deposition of one or more materials, such as superconductors, dielectrics and/or metals. Depending on the selected material, these materials can be deposited using deposition processes such as chemical vapor deposition, physical vapor deposition (e.g., evaporation or sputtering), or epitaxial techniques, among other deposition processes. Processes for fabricating circuit elements described herein can entail the removal of one or more materials from a device during fabrication. Depending on the material to be removed, the removal process can include, e.g., wet etching techniques, dry etching techniques, or lift-off processes. The materials forming the circuit elements described herein can be patterned using known lithographic techniques (e.g., photolithography or e-beam lithography).

During operation of a quantum computational system that uses superconducting quantum circuit elements and/or superconducting classical circuit elements, such as the circuit elements described herein, the superconducting circuit elements are cooled down within a cryostat to temperatures that allow a superconductor material to exhibit superconducting properties. A superconductor (alternatively superconducting) material can be understood as material that exhibits superconducting properties at or below a superconducting critical temperature. Examples of superconducting material include aluminum (superconductive critical temperature of 1.2 kelvin) and niobium (superconducting critical temperature of 9.3 kelvin). Accordingly, superconducting structures, such as superconducting traces and superconducting ground planes, are formed from material that exhibits superconducting properties at or below a superconducting critical temperature.

In certain implementations, control signals for the quantum circuit elements (e.g., qubits and qubit couplers) may be provided using classical circuit elements that are electrically and/or electromagnetically coupled to the quantum circuit elements. The control signals may be provided in digital and/or analog form.

Computer-readable media suitable for storing computer program instructions and data include all forms of non-volatile digital and/or quantum memory, media and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks; and quantum systems, e.g., trapped atoms or electrons. It is understood that quantum memories are devices that can store quantum data for a long time with high fidelity and efficiency, e.g., light-matter interfaces where light is used for transmission and matter for storing and preserving the quantum features of quantum data such as superposition or quantum coherence.

Control of the various systems described in this specification, or portions of them, can be implemented in a computer program product that includes instructions that are stored on one or more non-transitory machine-readable storage media, and that are executable on one or more processing devices. The systems described in this specification, or portions of them, can each be implemented as an apparatus, method, or system that may include one or more processing devices and memory to store executable instructions to perform the operations described in this specification.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by a quantum computer, the method comprising:
preparing an initial quantum state, comprising preparing one or more Slater determinants of a fermionic system in first quantization;
performing a first quantized quantum algorithm on the initial quantum state to simulate time evolution of the fermionic system and generate a time evolved quantum state; and
measuring the time evolved quantum state.

2. The method of claim 1, wherein preparing the one or more Slater determinants of the fermionic system in first quantization comprises, for each Slater determinant:
generating a superposition of configurations of occupied orbitals in the Slater determinant, wherein electron registers storing labels of the occupied orbitals are sorted within each configuration according to a predetermined order; and
applying an anti-symmetrization procedure to the superposition of configurations.

3. The method of claim 2, wherein the predetermined order comprises ascending order.

4. The method of claim 1, wherein preparing the one or more Slater determinants of the fermionic system in first quantization comprises, for each Slater determinant:
initializing multiple particle registers of qubits and an ancilla register as zero;
for q=1 to N, where N represents a number of basis functions used to express each occupied orbital in the fermionic system:
performing a single step of unary iteration on each particle register;
adding a value in qubit q to the ancilla register;
using qubit q to control unary iteration on the ancilla register;
writing, using the unary iteration, a value q into the ancilla register;
converting control qubits in the ancilla register to one-hot unary;
for each particle register of the multiple particle registers, using the control qubits and an output from the unary iteration to control a NOT on qubit q; and
converting the control qubits from one-hot unary.

5. The method of claim 4, wherein writing the value q into the ancilla register comprises using CNOT gates.

6. The method of claim 4, wherein converting control qubits in the ancilla register to one-hot unary comprises using CNOT gates.

7. The method of claim 4, wherein converting the control qubits from one-hot unary comprises uses CNOT gates.

8. The method of claim 4, wherein preparing the one or more Slater determinants of the fermionic system in first quantization further comprises, for each Slater determinant, performing a sequence of Givens rotations on the qubits to convert the qubits to first quantization.

9. The method of claim 8, wherein performing the sequence of Givens rotations comprises performing layers of Givens rotations on respective qubits, wherein performing each layer of Givens rotations converts one qubit to first quantization.

10. The method of claim 1, wherein the fermionic system comprises a system of η identical fermions that occupy N>>η orbitals and the quantum state comprise a wavefunction on η registers of n=┌log N┐ qubits included in the quantum computer.

11. The method of claim 1, wherein measuring the time evolved quantum state comprises measuring the time evolved quantum state to obtain one or more reduced density matrices (RDMs) in first quantization.

12. The method of claim 1, further comprising observing the fermionic system to characterize one or more properties of the fermionic system, and wherein preparing one or more Slater determinants of a fermionic system in first quantization uses the characterized one or more properties.

13. A quantum computing apparatus comprising:
quantum computing hardware comprising:
a plurality of physical qubits; and
control electronics configured to operate the plurality of qubits; and
a classical processor configured to receive and process data received from the quantum computing hardware;
wherein the quantum computing apparatus is configured to perform operations comprising:
preparing an initial quantum state, comprising preparing one or more Slater determinants of a fermionic system in first quantization;
performing a first quantized quantum algorithm on the initial quantum state to simulate time evolution of the fermionic system and generate a time evolved quantum state; and
measuring the time evolved quantum state.

14. The quantum computing apparatus of claim 13, wherein the plurality of physical qubits comprises η registers of n=┌log N┐ qubits, wherein η represents a number of identical fermions that occupy N>>η orbitals in a fermionic system to be simulated.

15. A method performed by a quantum computer, the method comprising:
converting an arbitrary quantum state of a quantum system in second quantization to a corresponding quantum state in first quantization, comprising preparing one or more Slater determinants of the quantum system in first quantization.

16. The method of claim 15, wherein preparing the one or more Slater determinants of the quantum system in first quantization comprises, for each Slater determinant:
generating a superposition of configurations of occupied orbitals in the Slater determinant, wherein electron registers storing labels of the occupied orbitals are sorted within each configuration according to a predetermined order;
applying an anti-symmetrization procedure to the superposition of configurations.

17. The method of claim 15, wherein preparing the one or more Slater determinants in first quantization comprises, for each Slater determinant:
initializing multiple particle registers of qubits and an ancilla register as zero;
for q=1 to N, where N represents a number of basis functions used to express each occupied orbital in the quantum system:
performing a single step of unary iteration on each particle register;
adding a value in qubit q to the ancilla register;
using qubit q to control unary iteration on the ancilla register;
writing, using the unary iteration, a value q into the ancilla register;
converting control qubits in the ancilla register to one-hot unary;
for each particle register of the multiple particle registers, using the control qubits and an output from the unary iteration to control a NOT on qubit q; and
converting the control qubits from one-hot unary.

18. The method of claim 17, further comprising performing a sequence of Givens rotations on the qubits to convert the qubits to first quantization.

19. The method of claim 18, wherein performing the sequence of Givens rotations comprises performing layers of Givens rotations on respective qubits, wherein performing each layer of Givens rotations converts one qubit to first quantization.

20. A quantum computing apparatus comprising:
quantum computing hardware comprising:
a plurality of physical qubits; and
control electronics configured to operate the plurality of qubits; and
a classical processor configured to receive and process data received from the quantum computing hardware;
wherein the quantum computing apparatus is configured to perform operations comprising converting an arbitrary quantum state of a quantum system in second quantization to a corresponding quantum state in first quantization, comprising preparing one or more Slater determinants of the quantum system in first quantization.

21. The quantum computing apparatus of claim 20, wherein the plurality of physical qubits comprises η registers of n=┌log N┐ qubits, wherein η represents a number of identical fermions that occupy N>>η orbitals in a fermionic system to be simulated.

22. A method performed by a quantum computer, the method comprising:
preparing an initial quantum state;
performing a first quantized quantum algorithm on the initial quantum state to simulate time evolution of a fermionic system and generate a time evolved quantum state; and
measuring, by the quantum computer, the time evolved quantum state to obtain one or more reduced density matrices (RDMs) in first quantization.

23. The method of claim 22, wherein measuring the time evolved quantum state to obtain one or more one-particle reduced density matrices in first quantization comprises performing a classical shadows method that applies separate random Clifford channels to each qubit register of multiple qubit registers that represent respective occupied orbitals in the fermionic system.

24. The method of claim 22, wherein the time evolved state comprises a first quantized state and wherein measuring the time evolved quantum state to obtain the one or more RDMs in first quantization comprises:
computing a classical shadow of the time evolved quantum state using an ensemble comprising a tensor product of copies of a uniform distribution over qubit Clifford circuits.

25. The method of claim 24, wherein measuring the time evolved quantum state to obtain the one or more RDMs in first quantization further comprises:
determining a number of samples to include in the classical shadow;
sampling, the determined number of times, from the ensemble to obtain multiple unitary operators;
evaluating, for each sample, expectation values of terms in a sum over a set of permutations of multiple qubit registers that represent respective occupied orbitals in the fermionic system;
grouping the samples into multiple groups of a same size;
averaging within each groups; and
computing a median of the averages to obtain a final estimate.

26. The method of claim 25, wherein evaluating the expectation values of the terms in the sum comprises using a Gottesman-Knill theorem.

27. The method of claim 25, wherein the multiple groups comprise
$$8 log ( \frac{1}{δ} )$$
groups, wherein δ represents a predefined target probability of accuracy.

28. The method of claim 25, wherein the same size is equal to b=4 Var(\hat{d})/ϵ², wherein ϵ represents a predefined additive error and \hat{d} represents an estimator for a k-RDM.

29. The method of claim 28, wherein the estimator for the k-RDM is given by
$$\hat{d} = \frac{{k}^{k} ( η ! )}{{η}^{k} ( η - k ) !} {\sum }_{x ∈ {R}_{k}} Tr [ \hat{ρ} {\prod }_{l = 1}^{k} {\vert {i}_{l} {j}_{l} \vert}_{{x}_{l}} ] ,$$
wherein η represents a number of identical fermions in the fermionic system, Rₖ represents the sum over the set of permutations of the multiple registers, Tr represents a trace operator, \hat{ρ} represents the classical shadow, and |iₗ [symbol] [symbol] jₗ|ₓ_{ₗ} represents a tensor product of |iₗ [symbol] [symbol] jₗ| on a l-th register in an xₗ element of the sum over the set of permutations with an identity operator on other registers.

30. The method of claim 25, wherein determining the number of samples to include in the classical shadow comprises computing 64e³ log (N/δ)k(2k+2e)^{k}η^{k}ϵ⁻² where e represents Euler's number, N represents a number of fermionic orbitals in the fermionic system, 1-δ represents a predefined target probability of accuracy, k represents the size of the RDMs, η represents the number of registers, and ϵ represents a predefined additive error.

31. The method of claim 22, wherein the measuring is performed in the computational basis.

32. The method of claim 22, wherein the RDMs comprise 1-RDMS and wherein to estimate the 1-RDM elements to within a predefined additive error ϵ, measuring the time evolved quantum state comprises performing a number of measurements of the order
$$\tilde{O} ( \frac{η}{{ϵ}^{2}} ) .$$

33. The method of claim 22, wherein the RDMs comprise k-RDMS and wherein to estimate the k-RDM elements to within a predefined additive error ϵ, measuring the time evolved quantum state comprises performing a number of measurements is of the order
$$\tilde{O} ( \frac{{k}^{k} {η}^{k}}{{ϵ}^{2}} ) .$$

34. The method of claim 22, wherein the fermionic system comprises a system of η identical fermions that occupy N>>η orbitals and the quantum state comprise a wavefunction on η registers of n=┌log N┐ qubits included in the quantum computer.

35. The method of claim 22, wherein the initial quantum state is prepared based on one or more observed properties of the fermionic system.

36. A quantum computing apparatus comprising:
quantum computing hardware comprising:
a plurality of physical qubits; and
control electronics configured to operate the plurality of qubits; and
a classical processor configured to receive and process data received from the quantum computing hardware;
wherein the quantum computing apparatus is configured to perform operations comprising:
preparing an initial quantum state;
performing a first quantized quantum algorithm on the initial quantum state to simulate time evolution of a fermionic system and generate a time evolved quantum state; and
measuring, by the quantum computer, the time evolved quantum state to obtain one or more reduced density matrices (RDMs) in first quantization.

37. The quantum computing apparatus of claim 36, wherein the plurality of physical qubits comprises η registers of n=┌log N┐ qubits, wherein η represents a number of identical fermions that occupy N>>η orbitals in a fermionic system to be simulated.

38. A method performed by a quantum computer, the method comprising:
measuring properties of a fermionic system wavefunction prepared using a first quantized algorithm, comprising applying separate random Clifford channels to each of qubit register of multiple qubit registers that represent occupied orbitals in the fermionic system.

39. The method of claim 38, wherein the time evolved state comprises a first quantized state and wherein measuring the time evolved quantum state to obtain the one or more RDMs in first quantization comprises:
computing a classical shadow of the time evolved quantum state using an ensemble comprising a tensor product of copies of a uniform distribution over qubit Clifford circuits.

40. The method of claim 39, wherein measuring the time evolved quantum state to obtain the one or more RDMs in first quantization further comprises:
determining a number of samples to include in the classical shadow;
sampling, the determined number of times, from the ensemble to obtain multiple unitary operators;
evaluating, for each sample, expectation values of terms in a sum over a set of permutations of the multiple registers;
grouping the samples into multiple groups of a same size;
averaging within each groups; and
computing a median of the averages to obtain a final estimate.

41. The method of claim 40, wherein determining the number of samples to include in the classical shadow comprises computing 64e³ log (N/δ)k(2k+2e)^{k}η^{k}ϵ⁻² where e represents Euler's number, N represents a number of fermionic orbitals in the fermionic system, 1-δ represents a predefined target probability of accuracy, k represents the size of the RDMs, η represents the number of registers, and ϵ represents a predefined additive error.

42. A quantum computing apparatus comprising:
quantum computing hardware comprising:
a plurality of physical qubits; and
control electronics configured to operate the plurality of qubits; and
a classical processor configured to receive and process data received from the quantum computing hardware;
wherein the quantum computing apparatus is configured to perform operations comprising measuring properties of a fermionic system wavefunction prepared using a first quantized algorithm, comprising applying separate random Clifford channels to each of qubit register of multiple qubit registers that represent occupied orbitals in the fermionic system.

43. The quantum computing apparatus of claim 42, wherein the plurality of physical qubits comprises η registers of n=┌log N┐ qubits, wherein η represents a number of identical fermions that occupy N>>η orbitals in a fermionic system to be simulated.
